(19) United States
(12) Patent Application Publication
(10) Pub. No.: US 2017/0296088 A1
(43) Pub. Date: Oct. 19, 2017

(54) ELECTRONIC DEVICE FOR MEASURING BIOMETRIC INFORMATION AND DEVICE FOR CHARGING THE ELECTRONIC DEVICE

(71) Applicant: Samsung Electronics Co., Ltd., Gyeonggi-do (KR)

(72) Inventor: Bongsuk CHOI, Seoul (KR)

(21) Appl. No.: 15/469,673

(22) Filed: Mar. 27, 2017

(30) Foreign Application Priority Data
Apr. 15, 2016 (KR) 10-2016-0046230

(51) Int. Cl. A61B 5/0408; A61B 5/00; A61B 5/0205; H05K 5/00; A61B 5/145; A61B 5/16; A61B 5/053; A61B 5/021; A61B 5/024; A61B 5/026

(52) U.S. Cl. CPC A61B 5/04085; A61B 5/7271; A61B 5/742; A61B 5/02055; H05K 5/0017; A61B 5/14542; A61B 5/165; A61B 5/6801; A61B 5/4806; A61B 5/021; A61B 5/024; A61B 5/026; A61B 5/0533; A61B 2560/0214; A61B 2560/0242; A61B 2562/166

(57) ABSTRACT

Various embodiments of the present disclosure relate to an electronic device for measuring biometric information and a device for charging the electronic device. The electronic device may include a housing having a top surface and a bottom surface, a first electrode disposed on the top surface of the housing, the first electrode electrically isolated from rest of the top surface, a second electrode disposed on the bottom surface of the housing and contacting a user's body when the electronic device is worn by the user, a sensor module electrically coupled to the first electrode and the second electrode, a processor electrically coupled to the sensor module, a display module electrically coupled to the processor; and a memory electrically coupled to the processor. The memory can store instructions that, when executed by the processor, causes the sensor module to measure the user's bio-signal using the first electrode and the second electrode, and the processor to provide the user's health state information by analyzing the measured bio-signal. Further, various embodiments can be implemented on the basis of the technical spirit and scope of the present disclosure.

### CLAIM OF PRIORITY

This application claims the benefit under 35 U.S.C. §119(a) of a Korean patent application filed on Apr. 15, 2016 in the Korean intellectual property office and assigned serial number 10-2016-0046230, the entire disclosure of which is hereby incorporated by reference.

### TECHNICAL FIELD

Various embodiments of the present disclosure relate to an electronic device for measuring biometric information and a device for charging the electronic device.

### BACKGROUND

Recently, in accordance with dramatic developments in technology, significant changes have been happening in the areas of medical diagnostics. For example, in the past, to properly measure a person's vitals, a trip to the doctor or hospital is often required. But now, healthcare-related medical equipment is now widely available, and users can obtain their vitals such as their blood sugar and blood pressure without visiting a hospital.

Meanwhile, development and interest in wearable electronic devices are increasing. Generally, an electronic device may require a plurality of electrodes to measure biometric information (biometric data). For example, an electrode must be held in each hand to obtain an electrocardiogram. Therefore, conventional electronic devices may include 2 electrodes that, when touches by the user, obtain measurements for an electrocardiogram. In this way, conventional electronic devices are inconvenient to the user because generally, the two electrodes must be held in each hand. Further, conventional electronic devices are inconvenient for measuring different types of biometric information (e.g., heart rate, degree of oxygen saturation (saturation pulse oximetry O2: SPO2)) because generally each biometric information requires a separate sensor.

In addition, conventional electronic devices need to be connected to a separate charging device in order to charge the electronic device. This is generally done by placing the electronic device in a cradle. In doing so, a male connector of the cradle must be inserted into a female connector of the electronic device.

### SUMMARY

The electronic device according to one embodiment of the present disclosure may use one of the electrode used to detect biometric data as a charging terminal and therefore does not require a separate charging terminal and a separate coupling structure.

An electronic device according to one embodiment of the present disclosure may include a housing having a top surface and a bottom surface, a first electrode disposed on the top surface of the housing, the first electrode electrically isolated from rest of the top surface, a second electrode disposed on the bottom surface of the housing and contacting a user's arm when the electronic device is worn by the user, wherein the first electrode is configured to be contacted by a portion of the user's other arm or hand when the electronic device is worn by the user, a sensor module electrically coupled to the first electrode and the second electrode, a processor electrically coupled to the sensor module, a display module electrically coupled to the processor; and a memory electrically coupled to the processor. The memory can store instructions that, when executed by the processor, causes the sensor module to measure the user's bio-signal using the first electrode and the second electrode, and the processor to provide the user's health state information by analyzing the measured bio-signal.

Further, an external electronic device according to one embodiment of the present disclosure may include a housing, two contact pins exposed to an exterior of the housing and configured to contact the second electrode, at least one magnetic member disposed in the housing and provides an attracting force to the second electrode, an interface unit connected to a charging device for charging the electronic device and a printed circuit board coupled to the interface unit and the contact pin.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1A illustrates an electronic device according to an embodiment of the present disclosure;

FIG. 1B is an exploded perspective view illustrating an electronic device according to an embodiment of the present disclosure;

FIG. 2 illustrates a first housing of an electronic device according to an embodiment of the present disclosure;

FIG. 3A illustrates a structure for coupling a first electrode according to an embodiment of the present disclosure;

FIG. 3B illustrates various examples of a first electrode according to an embodiment of the present disclosure;

FIG. 4 illustrates a structure for coupling a first heartbeat sensor module according to an embodiment of the present disclosure;

FIG. 5 illustrates a structure for coupling a second electrode according to an embodiment of the present disclosure;

FIG. 6 illustrates a structure for coupling a second heartbeat sensor module according to an embodiment of the present disclosure;

FIG. 7 illustrates a structure for coupling a temperature sensor module according to an embodiment of the present disclosure;

FIG. 8 illustrates a structure for coupling a light/color sensor, such as an Red, Green, Blue (RGB) sensor module according to an embodiment of the present disclosure;

FIG. 9 illustrates a method for measuring a bio-signal by an electronic device according to an embodiment of the present disclosure;

FIG. 10 is a block diagram illustrating a configuration of an electronic device according to an embodiment of the present disclosure;

FIG. 11 is a flowchart illustrating a method for operating an electronic device according to an embodiment of the present disclosure;

FIG. 12 is an exploded perspective view illustrating a charging device according to an embodiment of the present disclosure; and

FIG. 13 is a cross-sectional view illustrating a state of coupling an electronic device to a charging device according to an embodiment of the present disclosure.

### DETAILED DESCRIPTION

The following description, with reference to the accompanying drawings, is provided to assist one of skill in a comprehensive understanding of embodiments of the present disclosure as defined by the claims and their equivalents. It includes various specific details to assist in that understanding but are to be regarded as merely exemplary. Accordingly, those of ordinary skill in the art will recognize that various changes and modifications of the embodiments described herein may be made without departing from the scope and spirit of the present disclosure. In addition, descriptions of well-known functions and constructions may be omitted for clarity and conciseness.

Expressions such as “include” and “may include” which may be used in the present disclosure denote the presence of the disclosed functions, operations, and constituent elements and do not limit one or more additional functions, operations, and constituent elements. In the present disclosure, terms such as “include” and/or “have” may be construed to denote a certain characteristic, number, step, operation, constituent element, component or a combination thereof, but may not be construed to exclude the existence of, or a possibility of, the addition of one or more other characteristics, numbers, steps, operations, constituent elements, components or combinations thereof.

Furthermore, in the present disclosure, the expression “and/or” includes any and all combinations of the associated listed words. For example, the expression “A and/or B” may include A, may include B, or may include both A and B.

In the present disclosure, expressions including ordinal numbers, such as “first” and “second,” etc., may modify various elements. However, such elements are not limited by the above expressions. For example, the above expressions do not limit the sequence and/or importance of the elements. The above expressions are used merely for the purpose to distinguish an element from the other elements. For example, a first user device and a second user device indicate different user devices although both of them are user devices. For example, a first element may be referred to as a second element, and similarly, a second element may also be referred to as a first element without departing from the scope of the present disclosure.

In the case where a component is referred to as being “connected” to, or “accessed” by another component, it should be understood that not only is the component directly connected to or accessed by the other component, but there may also exist another component between them. Meanwhile, in the case where a component is referred to as being “directly connected” or “directly accessed” to another component, it should be understood that there is no component therebetween.

The terms used in the present disclosure are only used to describe specific embodiments, and do not limit the present disclosure. As used herein, the singular forms are intended to include the plural forms as well, unless the context clearly indicates otherwise.

In this disclosure, an electronic device may be a device that involves a communication function. For example, an electronic device may be a smart phone, a tablet personal computer (PC), a mobile phone, a video phone, an e-book reader, a desktop PC, a laptop PC, a netbook computer, a personal digital assistant (PDA), a portable multimedia player (PMP), an MP3 player, a portable medical device, a digital camera, or a wearable device (e.g., a head-mounted device (HMD)) such as electronic eyeglasses, electronic clothes, an electronic bracelet, an electronic necklace, an electronic appcessory, or a smart watch).

The teachings of this disclosure may also apply to a smart home appliance that involves a communication function. For example, an electronic device may be a TV, a digital video disk (DVD) player, audio equipment, a refrigerator, an air conditioner, a vacuum cleaner, an oven, a microwave, a washing machine, an air cleaner, a set-top box, a TV box (e.g., Samsung HomeSync™, Apple TV™, Google TV™, etc.), a game console, an electronic dictionary, an electronic key, a camcorder, or an electronic picture frame.

The teachings of this disclosure may also apply to various medical devices (for example, various kinds of portable medical measuring device (blood glucose meter, heart rate meter, blood pressure meter, or a temperature measuring instrument, etc.), magnetic resonance angiography (MRA), magnetic resonance imaging (MRI), computed tomography (CT), camcorder, etc., navigation (navigation) devices, global positioning system receiver (GPS) receiver, EDR (event data recorder), flight data recorder (FDR), automotive infotainment (infotainment) devices, marine electronic equipment (e.g., marine navigation systems, gyrocompass, etc.), aviation electronics (avionics), security devices, automotive head unit (head unit), industrial or household robots, financial institutions, automatic teller machine (ATM), point of sales (POS) terminals, or Internet of Things (IoT) devices (e.g. light bulbs, various sensors, electric or gas meters, sprinkler systems, fire alarms, thermostat, street lights, toaster, fitness equipment, hot water tank, a heater, a boiler, etc.).

Hereinafter, for convenience in the description, an electronic device according to various embodiments of the present disclosure will be described on a smart watch as a wearable electronic device. However, the electronic device according to various embodiments of the present disclosure is not limited to the smart watch, and it can be provided as a bracelet, ring, and ankle bracelet.

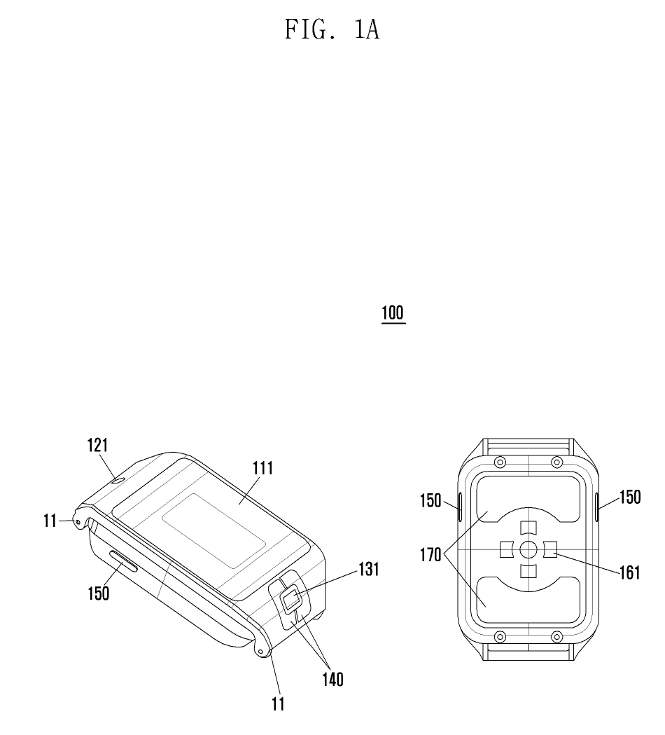

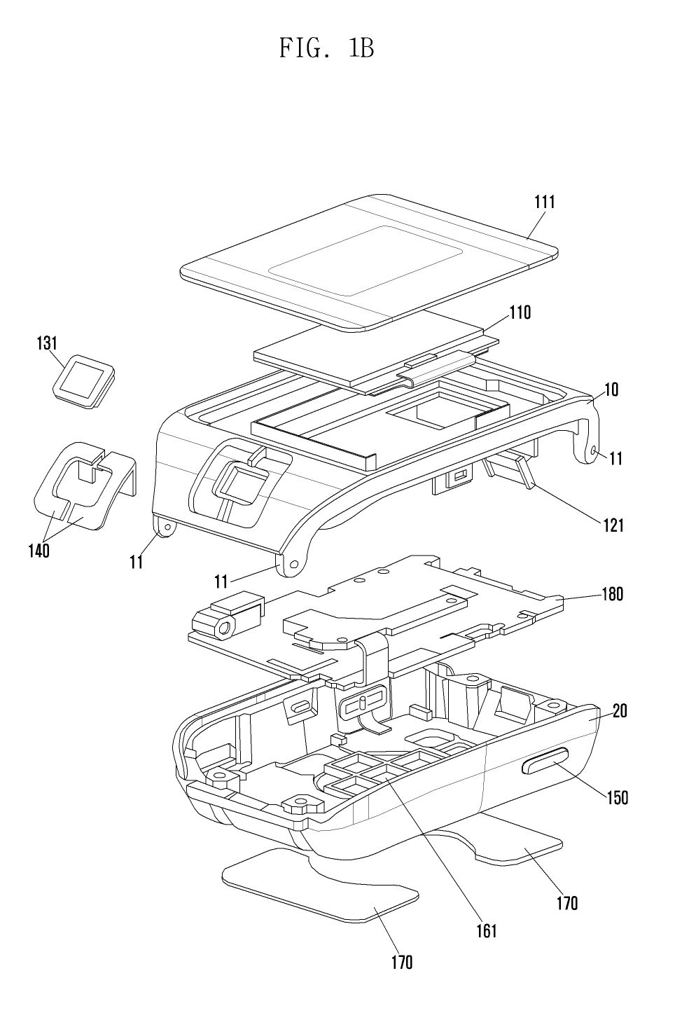

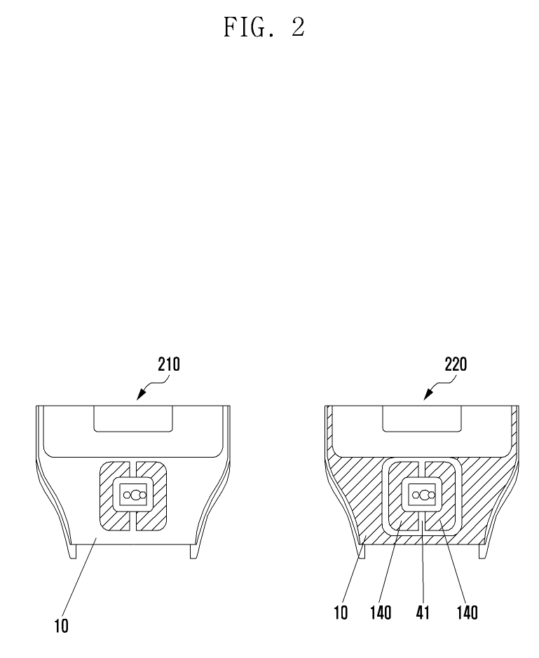

FIG. 1A illustrates an electronic device according to an embodiment of the present disclosure, FIG. 1B is an exploded perspective view illustrating an electronic device according to an embodiment of the present disclosure, and FIG. 2 illustrates a first housing of an electronic device according to an embodiment of the present disclosure.

With reference to FIGS. 1A to 2, the electronic device 100 according to an embodiment of the present disclosure may include a plurality of sensors (e.g., a RGB sensor, a Heart Rate Monitor (HRM) sensor, and/or a temperature sensor) and a plurality of electrodes (e.g., electrocardiogram (ECG) electrode and galvanic skin response (GSR) electrode) for measuring a user's health state (e.g., heart rate, heart electrical activity, pulse, blood pressure, body fat, sleep, stress, body temperature, biological age, body fatness, and total body fluid). The plurality of sensors and electrodes may be located by dividing a housing of the electronic device 100 into an upper surface and a rear surface. Further, the electronic device 100 may include at least one function key 150 at its side surface. The at least one function key 150 may include a power key, menu key, and selection key. Although FIG. 1A illustrates an electronic device 100 including 2 function keys 150, the electronic device 100 may include only one function key or more than 2 function keys. Further, the electronic device 100 may include a display module 110 for providing information related to a user's health state based on the user's bio-signals collected by the plurality of sensors and the plurality of electrodes. According to another embodiment, the display module 110 may include a touch panel.

The housing of the electronic device 100 may be configured with a first housing 10 as a front case and a second housing 20 as a rear case.

The display module 110 and a first window 111 protecting the display module 110 can be combined with the front (upper) surface of the first housing 10, a second window 121 protecting an RGB sensor (not shown) can be combined with a side surface (e.g., left surface) of the first housing 10, and a third window 131 protecting a first heart rate sensor module (not shown) and a first electrode 140 can be disposed on the lower portion of the first housing 10. A connecting part 11 for connecting a strap may be formed at the ends of the upper portion and the lower portion of the first housing 10.

The first housing 10 may be formed with plastic injection as shown by reference number 210 of FIG. 2. Alternatively, the first housing 10 may be made of a metallic material as shown by reference number 220 of FIG. 2. Here, the first electrode 140 can be electrically insulated from the first housing 10 when the first housing 10 is metallic. For example, an insulation material 41 (e.g., plastic injection) can be located between the first electrode 140 and the first housing 10 when the first housing 10 is metallic.

The first electrode 140 may be made of an electrically conductive material surrounding a third window 131. As shown in FIG. 2, the first electrode 140 may appear decorative. For example, the first electrode 140 may be made of stainless steel. Therefore, an embodiment of the present disclosure may integrate an electrode used for biometric sensing into the housing as a decorative design element.

The second housing 20 can be combined with the first housing 10. At least one function key 150 can be disposed on the left and/or right surface of the second housing 20, and a fourth window 161 protecting a second heart rate sensor module (not shown) and a second electrode 170 can be combined with the rear surface of the second housing 20.

A printed circuit board (PCB) 180 can be disposed in the housing by being placed between the first housing 10 and the second housing 20.

The plurality of sensors and electrodes (e.g., first electrode 140 and second electrode 170) can be electrically connected to the printed circuit board 180. For example, the first electrode 140 and the second electrode 170 can be connected electrically to a sensor module (not shown) installed on the printed circuit board 180 by using a first connecting element and a second connecting element (e.g., C-clip).

The electronic device 100 shown in FIGS. 1A to 2 is exemplary, and the embodiments of the present disclosure are not limited thereto. Although FIGS. 1A to 2 illustrate the first electrodes 140 as having two sub-electrodes, only one first electrode 140 can be formed according to another embodiment. Further, although the electronic device 100 is illustrated in a rectangular form, the electronic device 100 can be manufactured in a circular form according to another embodiment.

Hereinafter, a coupling structure for a plurality of sensors and electrodes is described in more detail.

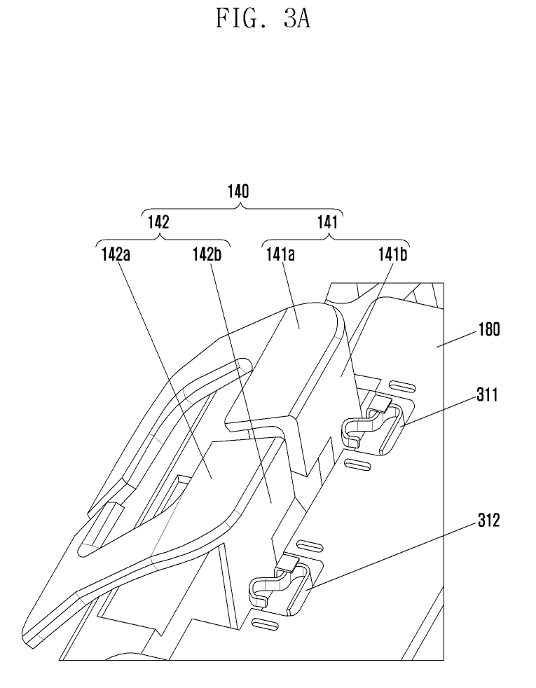

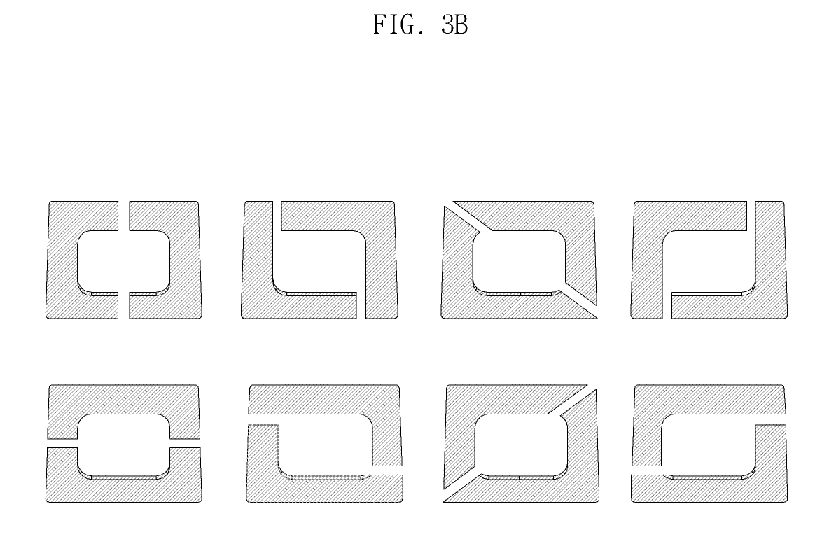

FIG. 3A illustrates a structure for coupling a first electrode according to an embodiment of the present disclosure, and FIG. 3B illustrates various examples of a first electrode according to an embodiment of the present disclosure.

With reference to FIGS. 3A and 3B, the first electrode 140 according to an embodiment of the present disclosure may be divided into 2 sub-electrodes. For example, the first electrode 140 can be divided into a first front electrode 141 and a second front electrode 142 by forming a separation at the center of the first electrode 140. The first front electrode 141 and the second front electrode 142 may substantially the same front area size. Meanwhile, according to another embodiment, the first electrode 140 can be separated in various forms as shown in FIG. 3B.

The first front electrode 141 and the second front electrode 142 may contact with a side surface of the printed circuit board 180. For example, the first front electrode 141 and the second front electrode 142 may include outer surfaces 141a and 142a and contact surfaces 141b and 142b extending from the outer surfaces 141a and 142a. The contact surfaces 141b and 142b may be connected to the printed circuit board 180. The contact surfaces 141b and 142b can be connected electrically to the printed circuit board 180 through first connecting elements 311 and 312. For example, the first connecting elements 311 and 312 can be C-clips having an elastic property as shown in FIG. 3A. However the present disclosure is not limited to this. For example, the first connecting elements 311 and 312 may be made with a sponge, Poron, or conductive adhesive tape. According to an embodiment, the contact surfaces 141b and 142b can contact directly with a contact pad (not shown) formed on the printed circuit board 180, or be connected to the printed circuit board 180 through an Flexible Printed Circuit Board (FPCB) or a cable. The first front electrode 141 and the second front electrode 142 can be connected to a sensor module (not shown) installed on the printed circuit board 180 through wirings of the printed circuit board.

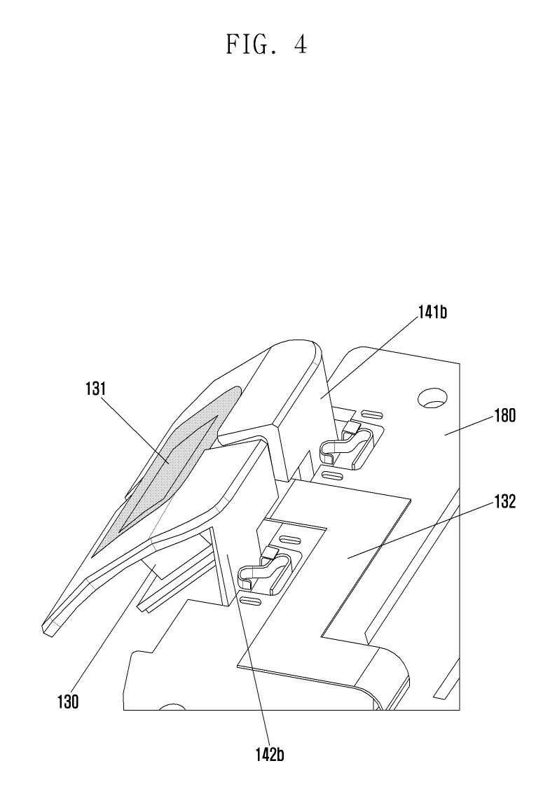

FIG. 4 illustrates a structure for coupling a first heartbeat sensor module according to an embodiment of the present disclosure.

With reference to FIG. 4, the first heart rate sensor module 130 according to an embodiment of the present disclosure may be located under the third window 131. The first heart rate sensor module 130 can be connected electrically to the printed circuit board 180 through an FPCB 132. The FPCB 132 can be connected to the printed circuit board 180 by passing through a slit formed between contact surfaces 141b and 142b of the first electrode 140.

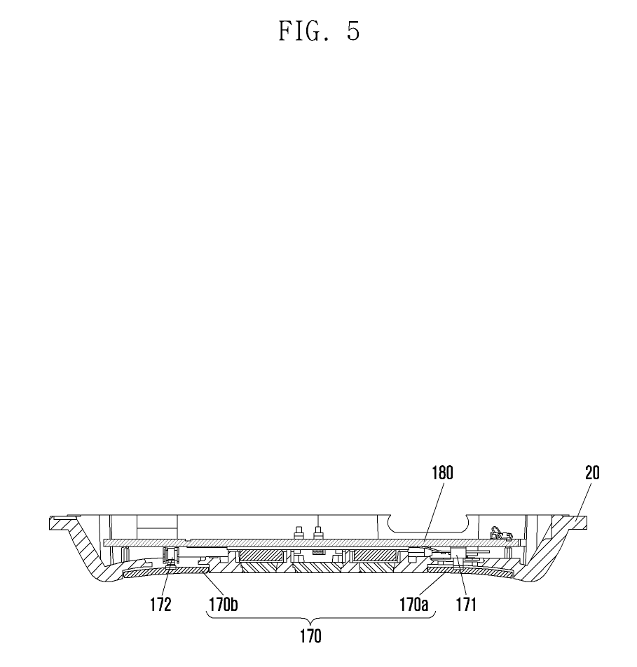

FIG. 5 illustrates a structure for coupling a second electrode according to an embodiment of the present disclosure.

With reference to FIG. 5, the second electrode 170 according to an embodiment of the present disclosure may be located at the bottom surface of the second housing 20. The second electrode 170 may be configured with two electrodes. For example, the second electrode 170 may include a first rear electrode 170a and a second rear electrode 170b.

The first rear electrode 170a and the second rear electrode 170b can be connected electrically to the printed circuit board 180 through second connecting elements 171 and 172. The second connecting elements 171 and 172 may be C-clips having an elastic property. However, the present disclosure is not limited to this. For example, the second connecting elements 171 and 172 can be made with a sponge, Poron, or conductive adhesive tape. According to another embodiment, the second electrode 170 can contact directly with a contact pad formed on the printed circuit board 180 (e.g., side surface), or can be connected to the printed circuit board 180 through an FPCB or a cable.

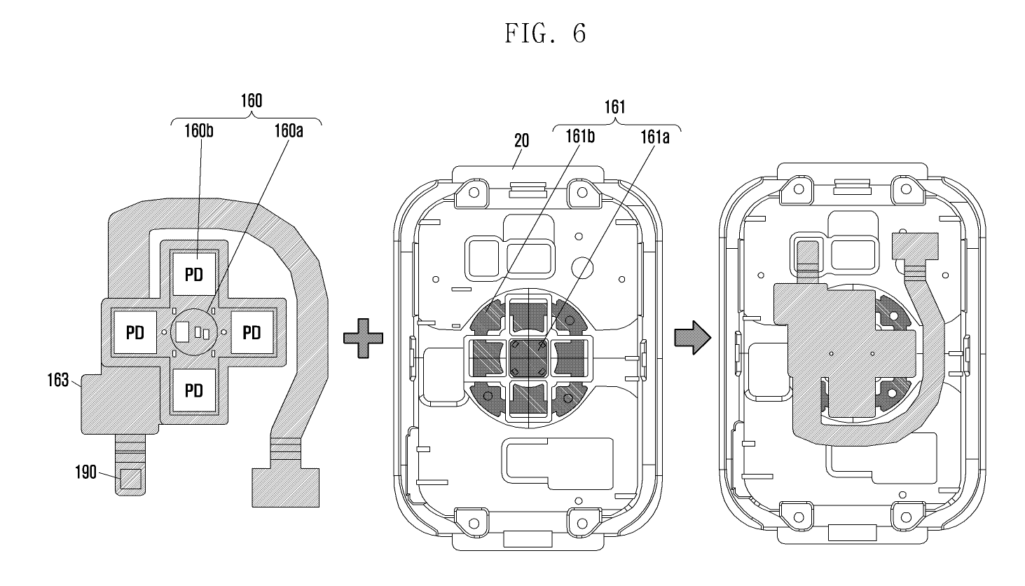

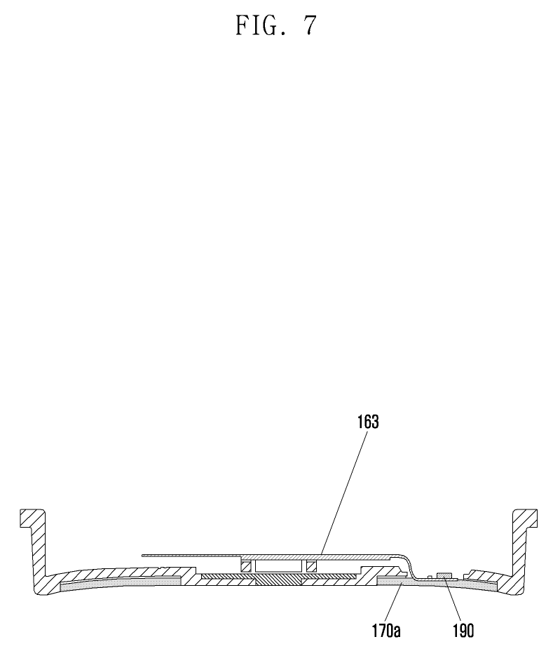

FIG. 6 illustrates a structure for coupling a second heartbeat sensor module according to an embodiment of the present disclosure, and FIG. 7 illustrates a structure for coupling a temperature sensor module according to an embodiment of the present disclosure.

With reference to FIGS. 6 and 7, the second heart rate sensor module 160 according to an embodiment of the present disclosure may be installed on a discrete FPCB. The second heart rate sensor module 160 may be configured with a light emitter 160a and a light receiver 160b. The light emitter 160a may be an LED (Light Emitting Diode), and the light receiver 160b may be a PD (Photo Diode).

The discrete FPCB 163 can be disposed on the second housing 20. Here, the second heart rate sensor module 160 can be covered by a fourth window 161. The fourth window 161 may be divided into a first part 161a covering the light emitter 160a and a second part 161b covering the light receiver 160b. The fourth window 161 may be divided into first part 161a and second part 161b by an opaque divider. This is done because the light generated by the light emitter 160a may be a noise source for the light receiver 160b. Therefore, to reduce the amount of light from light emitter 160a that is detected by the light receiver 160b, the fourth window 161 is divided into at least two parts by an opaque divider.

The fourth window 161 may have similar properties to those of the second housing 20 and may be formed with a material having an excellent adhesion power and high anti-scratch properties. For example, in case that the second housing 20 is made with a PC (polycarbonate) material, the fourth window 161 can be made with a transparent Durabio resin. The fourth window 161 and the second housing 20 can be combined by an insert injection method.

A temperature sensor 190 may be installed at a side of the discrete FPCB 163. The temperature sensor 190 may be located at one of the second electrodes 170. For example, the temperature sensor 190 can be attached to the interior surface of the first rear electrode 170a by using an adhesive tape as shown in FIG. 7. Various temperature sensors of contact or non-contact type can be used as the temperature sensor 190. For example, the temperature sensor 190 can be a temperature sensor that detects temperature using a thermistor or infrared ray. Such a temperature sensor 190 is well known to those skilled in the art; therefore, detailed description on the temperature sensor 190 is omitted here.

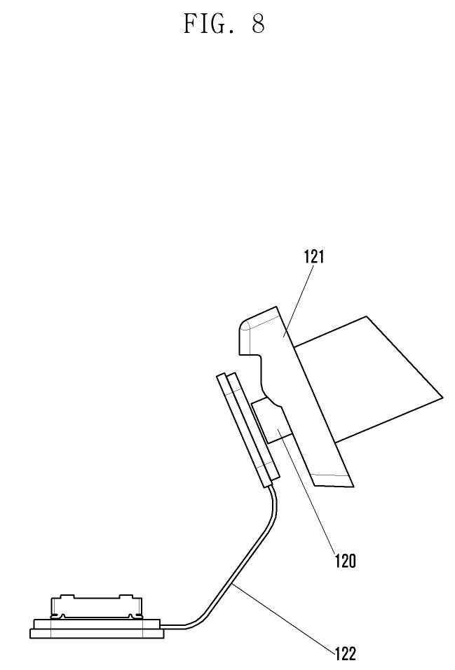

FIG. 8 illustrates a structure for coupling a light/color sensor such as an RGB sensor module according to an embodiment of the present disclosure.

With reference to FIG. 8, the RGB sensor 120 according to an embodiment of the present disclosure can be installed with the second window 121, and the RGB sensor 120 can be connected to the printed circuit board 180 through an FPCB 122.

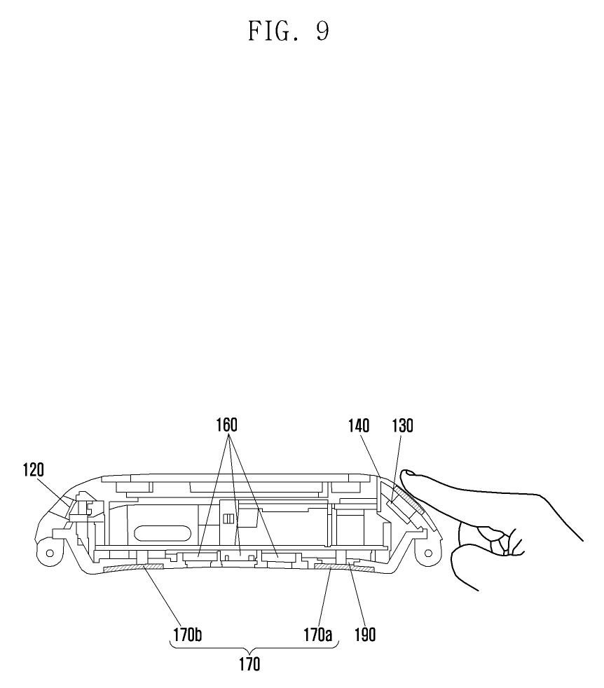

FIG. 9 illustrates a method for a bio-signal to be measured by an electronic device according to an embodiment of the present disclosure.

With reference to FIG. 9, the electronic device 100 according to one embodiment of the present disclosure can be worn on a user's body (e.g., on a wrist). If the user wears the electronic device 100, the second electrode 170, the second heart rate sensor module 160, and the temperature sensor 190 can be in contact with the user's body. Here, the electronic device 100 can measure a bio-signal through a plurality of sensors and electrodes to provide information related to a user's health state. For example, the electronic device 100 can measure a user's body temperature through the temperature sensor 190. Further, the electronic device 100 can measure bio-signals (e.g., galvanic skin response) through the rear electrodes 170a and 170b to analyze stress and emotion of the user. Further, the electronic device 100 can detect changes of blood pressure and flow through the second heart rate sensor module 160 by measuring the user's heart rate. Further, the electronic device 100 can measure an outdoor visibility (i.e. light intensity) of the surrounding environment using the RGB sensor 120. The RGB sensor can further detect blue light intensity for the purposes of performing an analysis of the user's sleep. Further, the electronic device 100 can measure an intensity of infrared rays through the first heart rate sensor module 130.

As shown in FIG. 9, if a finger of a user's hand not wearing the electronic device 100 touches the first heart rate sensor module 130, the electronic device 100 can measure a heart rate and a degree of oxygen saturation (saturation pulse oximetry O2: SPO2) through the first heart rate sensor module 130.

Further, if the user touches one of the first electrodes 140 with a hand not wearing the electronic device 100, the electronic device 100 can provide electrocardiogram information based on bio-signals measured by one of the first electrode 140 and the second electrode 170.

Further, if the user simultaneously touches both the first front electrode 141 and the second front electrode 142 with a hand not wearing the electronic device 100, the electronic device 100 can provide BIA (Bio-Impedance Analysis) information based on bio-signals measured by the first front electrode 141, second front electrode 142, and second electrode 170. The BIA indicates a hydration degree of tissue and an amount of body fluid, and from them body composition, body fluid balance, and cell health index can be known. For example, the electronic device 100 can provide a biological age, detoxification state, body fat percentage, total body fluid, critical symptoms (e.g., cancer or kidney dialysis symptoms), early detection of lymphedema, signs of metabolic disease (e.g., diabetes, high blood pressure, stroke, and arteriosclerosis) through BIA.

Further, if the user simultaneously touches one of the first electrodes 140 and the first heart rate sensor module 130 with a hand not wearing the electronic device 100, the electronic device 100 can provide blood pressure information based on bio-signals measured by one of the first electrode 140, first heart rate sensor module 130, and second electrode 170.

As shown in FIG. 9, an embodiment of the present disclosure enables easy measurement of health information (e.g. heart rate, oxygen saturation, heart electrical activity, bio-impedance, and blood pressure) with a single touch by disposing the second front electrode 142 on a bottom surface of the electronic device touching the user, and providing the first front electrode 141 and the first heart rate sensor module 130 at a location so that the first front electrode 141 and the first heart rate sensor module 130 can be touched by the user's arm that is not wearing the electronic device.

Accordingly, the electronic device 100 according to one or more embodiments of the present disclosure enables measurements of stress, emotion, heart rate, blood pressure, blood flow, etc. by using the second electrode 170 and the second heart rate sensor module 160 located at the rear surface of the electronic device 100 and enables measurements of heart electrical activity, bio-impedance, blood pressure, oxygen saturation, etc. by using the first electrode 140 and the first heart rate sensor module 130 located at the front surface of the electronic device 100 in conjunction with the second electrode 170 and the second heart rate sensor module 160 located at the rear surface of the electronic device 100.

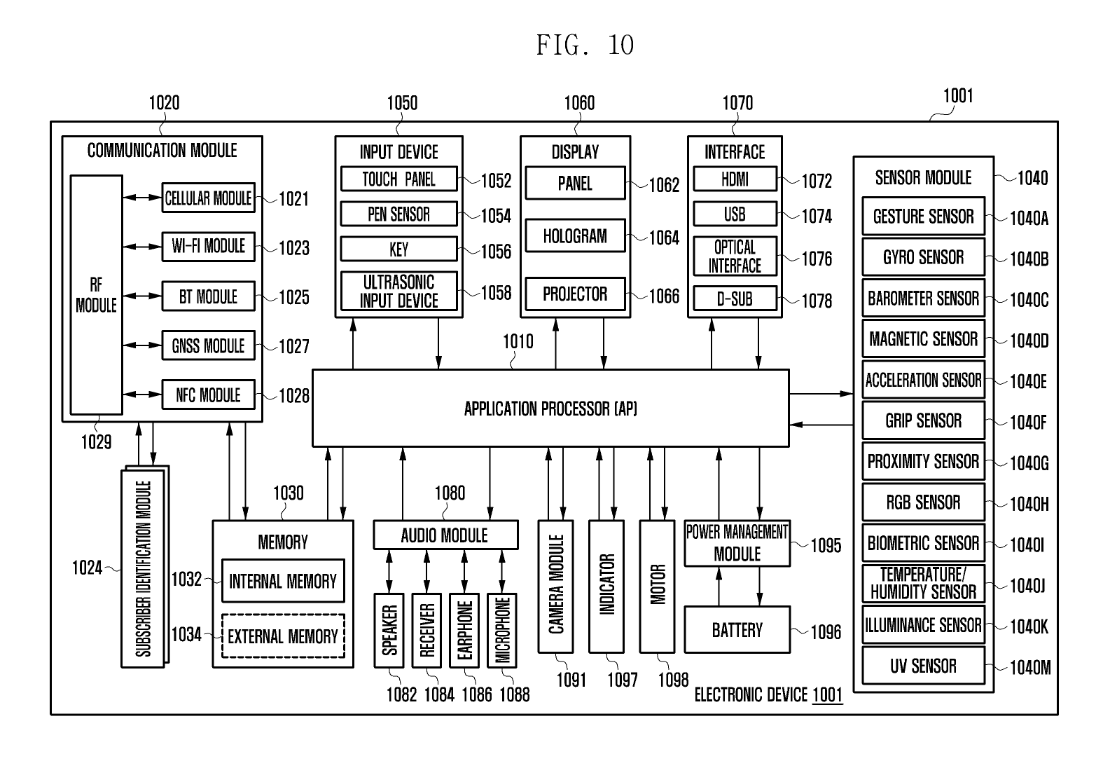

FIG. 10 is a block diagram illustrating an electronic device 1001 in accordance with an embodiment of the present disclosure. The electronic device 1001 may form, for example, the whole or part of the electronic device 100 shown in FIGS. 1 to 9. Referring to FIG. 10, the electronic device 1001 includes at least one application processor (AP) 1010, a communication module 1020, a subscriber identification module (SIM) card 1024, a memory 1030, a sensor module 1040, an input unit 1050, a display 1060, an interface 1070, an audio module 1080, a camera module 1091, a power management module 1095, a battery 1096, an indicator 1097, and a motor 1098.

The AP 1010 may drive an operating system or applications, control a plurality of hardware or software components connected thereto, and also perform processing and operation for various data including multimedia data. The AP 1010 may be a system-on-chip (SoC), for example. According to an embodiment of the present disclosure, the AP 1010 may further include a graphic processing unit (GPU). The AP 1010 may include a microprocessor or any suitable type of processing circuitry, such as one or more general-purpose processors (e.g., ARM-based processors), a Digital Signal Processor (DSP), a Programmable Logic Device (PLD), an Application-Specific Integrated Circuit (ASIC), a Field-Programmable Gate Array (FPGA), a Graphical Processing Unit (GPU), a video card controller, etc.

The communication module 1020 may include therein a cellular module 1021, a WiFi module 1023, a BT module 1025, a GNSS module 1027, an NFC module 1028, and an RF (radio frequency) module 1029.

The cellular module 1021 may offer a voice call, a video call, a message service, an internet service, and the like through a communication network (e.g., machine type communications (MTC), fifth generation (5G), long term evolution (LTE), long term evolution advanced (LTE-A), code division multiple access (CDMA), wideband code division multiple access (WCDMA), universal mobile telecommunications system (UMTS), wireless broadband (WiBro), global system for mobile communications (GSM), wireless fidelity (Wi-Fi), Bluetooth, and near field communications (NFC) etc.). Additionally, the cellular module 1021 may perform identification and authentication of the electronic device in the communication network, using the SIM card 1024. According to an embodiment of the present disclosure, the cellular module 1021 may perform at least part of functions the AP 1010 may provide. For example, the cellular module 1021 may perform at least part of a multimedia control function. Each of the WiFi module 1023, the BT module 1025, the GNSS module 1027 and the NFC module 1028 may include a processor for processing data transmitted or received. Although FIG. 10 shows the cellular module 1021, the WiFi module 1023, the BT module 1025, the GNSS module 1027 and the NFC module 1028 as different blocks, at least part of them may be contained in a single IC (integrated circuit) chip or a single IC package.

The RF module 1029 may transmit and receive data, e.g., RF signals or any other electric signals. The RF module 1029 may include a transceiver, a PAM (power amp module), a frequency filter, an LNA (low noise amplifier), and the like. Although FIG. 10 shows that the cellular module 1021, the WiFi module 1023, the BT module 1025, the GNSS module 1027 and the NFC module 1028 share the RF module 1029, at least one of them may perform transmission and reception of RF signals through a separate RF module.

The SIM card 1024 may include, for example, an embedded SIM including a user identification module, and may include unique identification information (e.g., an integrated circuit card identifier (ICCID)) or subscriber information (e.g., international mobile subscriber identity (IMSI)).

The memory 1030 includes an internal memory 1032 and an external memory 1034. The internal memory 1032 may include, for example, at least one of a volatile memory (e.g., a dynamic RAM (DRAM), a static RAM (SRAM), a synchronous dynamic RAM (SDRAM), etc.), and a non-volatile memory (e.g., a one time programmable ROM (OTPROM), a programmable ROM (PROM), an erasable and programmable ROM (EPROM), an electrically erasable and programmable ROM (EEPROM), a mask ROM, a flash ROM, a not AND (NAND) flash memory, a not OR (NOR) flash memory, etc.). According to an embodiment of the present disclosure, the internal memory 1032 may be in the form of a solid state drive (SSD). The external memory 1034 may further include a flash drive, for example, a compact flash (CF), a secure digital (SD), a micro-secure digital (micro-SD), a mini-secure digital (mini-SD), an extreme digital (xD), a memory stick, and the like. The external memory 1034 may be functionally connected to the electronic device 1001 through various interfaces.

The sensor module 1040 may measure physical quantity or sense an operating status of the electronic device 1001, and then convert measured or sensed information into electric signals. The sensor module 1040 includes, for example, at least one of a gesture sensor 1040A, a gyro sensor 1040B, an atmospheric sensor 1040C, a magnetic sensor 1040D, an acceleration sensor 1040E, a grip sensor 1040F, a proximity sensor 1040G, a color sensor 1040H (e.g., RGB (red, green, blue) sensor), a biometric sensor 1040I, a temperature-humidity sensor 1040J, an illumination sensor 1040K, and a UV (ultraviolet) sensor 1040M. Additionally or alternatively, the sensor module 1040 may include, e.g., an E-nose sensor, an EMG (electromyography) sensor, an EEG (electroencephalogram) sensor, an ECG (electrocardiogram) sensor, an IR (infrared) sensor, an iris scan sensor, or a finger scan sensor. Also, the sensor module 1040 may include a control circuit for controlling one or more sensors equipped therein.

The input unit 1050 includes a touch panel 1052, a digital pen sensor 1054, a key 1056, or an ultrasonic input unit 1058. The touch panel 1052 may recognize a touch input in a manner of capacitive type, resistive type, infrared type, or ultrasonic type. Also, the touch panel 1052 may further include a control circuit. The touch panel 1052 may further include a tactile layer. In this case, the touch panel 1052 may offer a tactile feedback to a user. The pen sensor 1054 (e.g., a digital pen sensor), for example, may be implemented by using a method identical or similar to a method of receiving a touch input from the user, or by using a separate sheet for recognition. For example, a key pad or a touch key may be used similar to the keys 1056. The ultrasonic input unit 1058 enables the terminal to sense a sound wave by using a microphone 1088 of the terminal through a pen generating an ultrasonic signal, and to identify data.

The display 1060 (e.g., the display 110) includes a panel 1062, a hologram 1064, or a projector 1066. The panel 1062 may have a flexible, transparent or wearable form. The panel 1062 may be formed of a single module with the touch panel 1052. The hologram 1064 may show a stereoscopic image in the air using interference of light. The projector 1066 may project an image onto a screen, which may be located at the inside or outside of the electronic device 1001. According to an embodiment of the present disclosure, the display 1060 may further include a control circuit for controlling the panel 1062, the hologram 1064, and the projector 1066.

The interface 1070 may include, for example, an HDMI (high-definition multimedia interface) 1072, a USB (universal serial Bus) 1074, an optical interface 1076, or a D-sub (D-subminiature) 1078. Additionally or alternatively, the interface 1070 may include, for example, an MHL (mobile high-definition link) interface, an SD (secure digital) card/MMC (multi-media card) interface, or an IrDA (infrared data association) interface.

The audio module 1080 may perform a conversion between sounds and electric signals. The audio module 1080 may process sound information inputted or outputted through a speaker 1082, a receiver 1084, an earphone 1086, or a microphone 1088.

The camera module 1091 is a device capable of obtaining still images and moving images. According to an embodiment of the present disclosure, the camera module 1091 may include at least one image sensor (e.g., a front sensor or a rear sensor), a lens, an ISP (image signal processor, not shown), or a flash (e.g., LED or xenon lamp, not shown).

The power management module 1095 may manage electric power of the electronic device 1001. The power management module 1095 may include, for example, a PMIC (power management integrated circuit), a charger IC, or a battery charge gauge. The PMIC may be implemented by, for example, an IC or an SoC semiconductor. Charging methods may be classified into a wired charging method and a wireless charging method. A wireless charging type may include, for example, a magnetic resonance type, a magnetic induction type, or an electromagnetic type. Any additional circuit for wireless charging may be further used such as a coil loop, a resonance circuit, or a rectifier. The battery gauge may measure the residual charge amount of the battery 1096 and a voltage, current or temperature in a charging process. The battery 1096 may store or create electric power therein and supply electric power to the electronic device 1001. The battery 1096 may be, for example, a rechargeable battery or a solar battery.

The indicator 1097 may show thereon a current status (e.g., a booting status, a message status, or a recharging status) of the electronic device 1001 or of its part (e.g., the AP 1010). The motor 1098 may convert an electric signal into a mechanical vibration. The electronic device 1001 may include a specific processor (e.g., GPU) for supporting a mobile TV. This processor may process media data that comply with standards of DMB (digital multimedia broadcasting), DVB (digital video broadcasting), or MediaFlo.

Each of the above-discussed elements of the electronic device disclosed herein may be formed of one or more components, and its name may vary according to the type of the electronic device. The electronic device disclosed herein may be made with at least one of the above-discussed elements without some other elements or with additional other elements. Some of the elements may be integrated into a single entity that still performs the same functions as those of such elements that may be separable.

The electronic device 1001 may further include a plurality of electrodes (e.g., first electrode 140 and second electrode 170 shown in FIGS. 1 to 9). Further, the biometric sensor 1040I may further include a temperature sensor for measuring a body temperature (e.g., temperature sensor 190 of FIGS. 6 and 7) and a heart rate sensor module (e.g., first heart rate sensor module 130 of FIG. 4 and second heart rate sensor module 160 of FIG. 6). Further, the sensor module 1040 of the electronic device 1001 may further include an RGB sensor module (e.g., RGB sensor 120 of FIG. 8).

The electronic device 1001 according to an embodiment of the present disclosure may include a plurality of electrodes and sensors distributed at the upper and lower parts of the electronic device 1001. The electronic device 1001 can provide a user's health information by analyzing bio-signals measured through the plurality of electrodes and sensors. Further, the electronic device 1001 can store the health information in the memory 1030 for a predetermined period, semi-permanently, or permanently. Further, the electronic device 1001 can transmit the user's health information periodically to a designated terminal or server. Further, if the user's health suddenly becomes worse, for example, if the user falls down suddenly, the electronic device 100 can transmit an emergency message to a designated terminal or an emergency center.

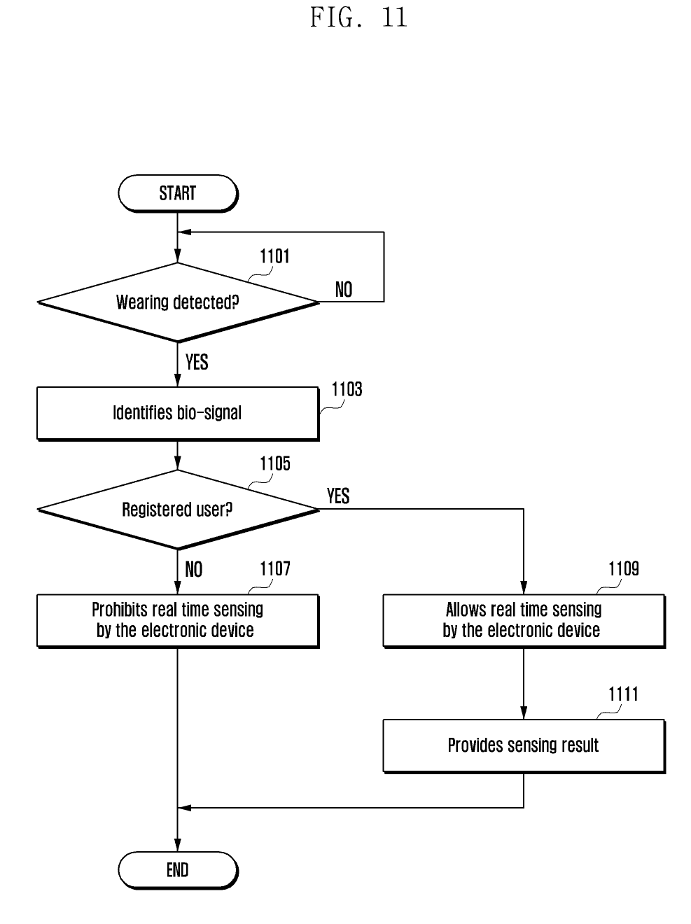

The processor 1010 of the electronic device 1001 can identify whether the electronic device 1001 is worn by a user. If the electronic device is identified as being worn on the user's body, the processor 1010 can identify whether the user is a registered user by checking a bio-signal (e.g., heart signal). Detailed descriptions on this will be made with reference to FIG. 11. FIG. 11 is a flowchart illustrating a method for operating an electronic device according to an embodiment of the present disclosure.

According to an embodiment of the present disclosure with reference to FIG. 11, a processor (e.g., processor 1010 of FIG. 10) of an electronic device (e.g., electronic device 100 of FIG. 1 or electronic device 1001 of FIG. 10) identifies at operation 1101 whether the electronic device is worn by a user. For example, the processor can identify whether the electronic device is being worn by a user by using a temperature sensor (e.g., reference number 190 of FIGS. 6 and 7). According to another embodiment, the processor can detect the wearing of an electronic device by using various sensor modules located at the bottom surface of the electronic device.

If the electronic device is identified as being worn by a user, the processor identifies a user's bio-signal (e.g., heart signal) at operation 1103. For example, the processor can identify a user's heart signal (e.g., by an ECG) by using bio-signals measured by the second electrode 170 and one of the first electrodes 140. According to another embodiment, the processor can output a message and/or a sound directing the user to touch a front electrode when wearing of the electronic device is detected.

The processor identifies whether the user is a registered user at operation 1105. For example, the processor can compare the identified bio-signal with a user's bio-signal stored previously in order to identify whether the user is a registered user.

If the user is identified as an unregistered user at operation 1105, at operation 1107 the processor prohibits real time sensing by the electronic device.

On the other hand, if the user is identified as a registered user at operation 1105, at operation 1109 the processor allows real time sensing by the electronic device, and provides a sensing result at operation 1111.

It would be recognized that when a general purpose computer accesses code for implementing the processing shown herein, the execution of the code transforms the general purpose computer into a special purpose computer for executing the processing shown herein. Any of the functions and steps provided in the Figures may be implemented in hardware, software or a combination of both and may be performed in whole or in part within the programmed instructions of a computer.

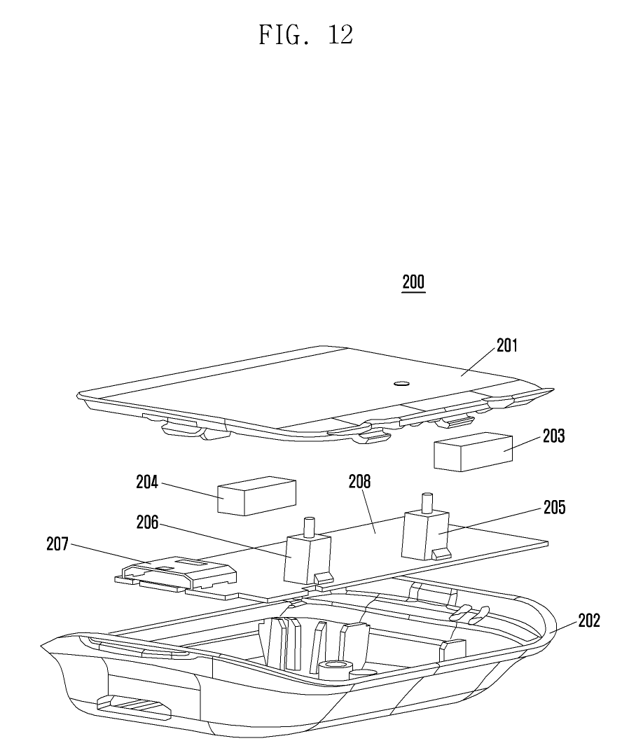

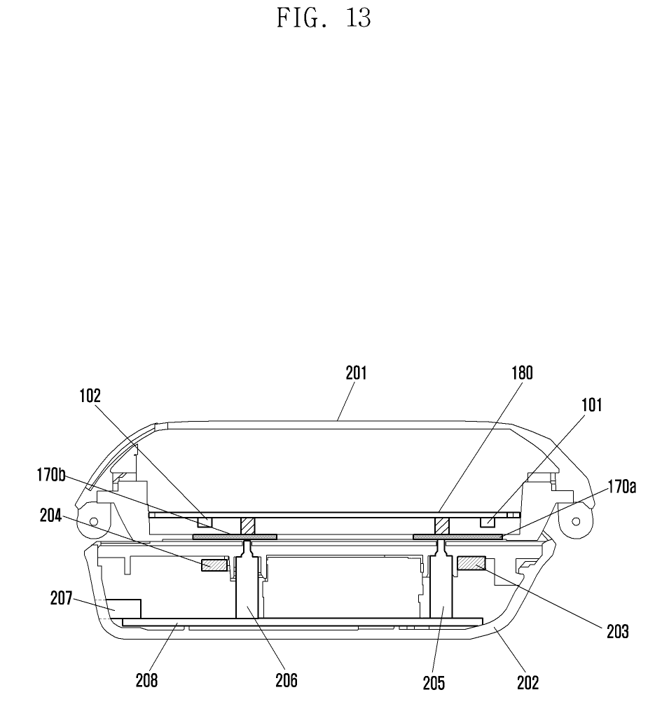

FIG. 12 is an exploded perspective view illustrating a charging device according to an embodiment of the present disclosure, and FIG. 13 is a cross-sectional view illustrating a state of coupling an electronic device to a charging device according to an embodiment of the present disclosure.

With reference to FIGS. 12 and 13, the electronic device 200 (hereinafter, second electronic device or the external device) according to an embodiment of the present disclosure may be an accessory device for charging the electronic device 100 (hereinafter, first electronic device).

The second electronic device 200 may include a first housing 201, second housing 202, first magnetic member 203, second magnetic member 204, first contact pin 205, second contact pin 206, interface unit 207, and printed circuit board 208. The first magnetic member 203 can be placed adjacent to the first contact pin 205, and the second magnetic member 204 can be placed adjacent to the second contact pin 206.

The first housing 201 may be a case covering the upper surface of the second electronic device 200, and the second housing 202 may be a case covering the lower surface and side surfaces of the second electronic device 200.

An second external device (e.g., the electric charger) can be connected to the interface unit 207. The interface unit 207 may be a connector having specifications of USB (Universal Serial Bus), micro-USB, and USB 3.0 C-type. The printed circuit board 208 can be installed to be connected to the interface unit 207, first contact pin 205, and second contact pin 206.

The first contact pin 205 and the second contact pin 206 may contact respectively with a first rear electrode 170a and a second rear electrode 170b of the first electronic device 100. The first contact pin 205 and the second contact pin 206 can transmit power from the electric charger (not shown) connected through the interface unit 207 to the first electronic device 100. For example, as shown in FIG. 13, the first contact pin 205 can be connected to a positive (+) electric terminal of the charger (not shown) through the interface unit 207, and the second contact pin 206 can be connected to a negative (−) electric terminal of the charger (not shown) through the interface unit 207. In turn, the first contact pin 205 is electrically coupled to the first rear electrode 170a and the second contact pin 206 to the second rear electrode 170b in order to transmit power from the electric charger to the first electronic device. Therefore, an embodiment of the present disclosure can use the first rear electrode 170a and the second rear electrode 170b for measuring bio-signals as well as for use as charging terminals. Accordingly, the first electronic device 100 does not require separate charging terminals.

Although FIGS. 12 and 13 illustrate that the first contact pin 205 and the second contact pin 206 may be pogo pins, but embodiments of the present disclosure are not so limited. For example, the first contact pin 205 and the second contact pin 206 can contact with the first rear electrode 170a and the second rear electrode 170b using various mechanism, for example C-clips.

According to an embodiment of the present disclosure, the first contact pin 205 and the second contact pin 206 can be installed at the right and left sides of the second electronic device 200 symmetrically. Accordingly, the second electronic device 200 can be coupled to the first electronic device 100 when the second electronic device 200 is variously oriented with respect to the first electronic device 100. According to another embodiment, the first contact pin 205 and the second contact pin 206 can be installed asymmetrically so as to distinguish the left side of the second electronic device 200 from the right side of the second electronic device 200. In doing so, the second electronic device 200 may be coupled to the first electronic device 100 only when the second electronic device 200 is oriented with respect to the first electronic device 100 in a particular direction.

The first magnetic member 203 and the second magnetic member 204 can be located between the first housing 201 and the second housing 202. The first magnetic member 203 and the second magnetic member 204 can be installed corresponding to the locations of the first rear electrode 170a and the second rear electrode 170b of the first electronic device 100. By this arrangement, the second electronic device 200 according to an embodiment of the present disclosure can be coupled to the first electronic device 100 without using a separate coupling structure because of attracting forces generated between the magnetic member 203 and 204 and the rear electrodes 170a and 170b. Further, the first electronic device 100, by virtue of already including the second electrode 170, does not require a separate magnetic or metallic member to be attracted to the first magnetic member 203 and the second magnetic member 204. The rear electrodes 170a and 170b of the first electronic device 100 may be made with NSSC 190 material from Nippon Steel & Sumikin Stainless or STS445NF material from POSCO.

The first electronic device 100 may further include a first sensor 101 and a second sensor 102 for identifying the first magnetic member 203 and the second magnetic member 204. The first sensor 101 and the second sensor 102 may be a Hall IC (Integrated Circuit) for identifying magnetic properties. The first sensor 101 and the second sensor 102 can be located corresponding to the locations of the first magnetic member 203 and the second magnetic member 204 respectively. Here, the magnetic property may include a polarity of a material, a magnetic field strength, and the like.

According to an embodiment of the present disclosure, the first electronic device 100 may include only one sensor (e.g., Hall IC). For example, the first electronic device 100 may include only one sensor (e.g., Hall IC) for detecting a coupling state when the first contact pin 205 and the second contact pin 206 are installed asymmetrically on the second electronic device 200, because it is unnecessary to identify a coupling direction if the first electronic device 100 and the second electronic device 200 are always coupled in the same orientation.

Meanwhile, if the coupling direction of the second electronic device 200 is not limited to a single orientation, the first electronic device 100 must identify the coupling direction of the second electronic device 200. This is because locations of contact pins being connected to the first rear electrode 170a and the second rear electrode 170b can change according to the coupling direction of the second electronic device 200. For example, as shown in FIG. 13, if the first electronic device 100 is coupled so that the first magnetic member 203 is located at the right side and the second magnetic member 204 is located at the left side, a positive (+) current may be supplied to the first rear electrode 170a of the first electronic device 100 by contacting the first contact pin 205 with the first magnetic member 203 and a negative (−) current may be supplied to the second rear electrode 170b by contacting the second contact pin 206 with the second magnetic member 204. Conversely, if the first electronic device is coupled so that the first magnetic member 203 is located at the left side and the second magnetic member 204 is located at the right side, a negative (−) current may be supplied to the first rear electrode 170a of the first electronic device 100 and a positive (+) currently may be supplied to the second rear electrode 170b. In that case, the electronic device 100 may not be able to accept power from the second electronic device 200. Therefore, the first electronic device 100 may include a switch (not shown) for connecting the first rear electrode 170a with a charging module (not shown) and connecting the second rear electrode 170b with a ground, or connecting the first rear electrode 170a with a ground and connecting the second rear electrode 170b with the charging module (not shown), according to the coupling direction of the second electronic device 200.

If the first magnetic member 203 and the second magnetic member 204 are installed in the second electronic device 200 symmetrically, an embodiment of the present disclosure can identify a coupling direction of the second electronic device 200 because the magnetic properties of the first magnetic member 203 and the second magnetic member 204 are different. For example, the first electronic device 100 can identify the coupling direction of the second electronic device 200 because the magnetic properties identified by the first sensor 101 and second sensor 102 are different, depending on the orientation of the second electronic device 200 to the first electronic device 100.

Alternatively, by changing the locations of first sensor 101 and the second sensor 102 for identifying the first magnetic member 203 and the second magnetic member 204, the first electronic device 100 can identify the coupling direction of the second electronic device 200. For example, the first electronic device 100 can identify the coupling direction of the second electronic device 200 because the magnetic properties (e.g. magnetic field strength) identified by the first sensor 101 and the second sensor 102 of the first electronic device 100 is different even though the first magnetic member 203 and the second magnetic member 204 have the same magnetic property.

Alternatively, the magnetic properties (e.g. magnetic field strength) identified by the first sensor 101 and the second sensor 102 of the first electronic device 100 can be different if the first magnetic member 203 and the second magnetic member 204 are installed asymmetrically.

The first electronic device 100 may further include a switch for connecting the second electrode 170 to a sensor module (not shown) or a charging module (not shown) according to the coupling state of the second electronic device 200. According to another embodiment, the first electronic device 100 may include a switch for blocking or connecting a path between the second electrode 170 and the sensor module (not shown) according to the coupling state of the second electronic device 200.

As described above, one or more embodiments of the present disclosure do not require a separate electrode because a conductive portion of the housing, which may be seen as decorative, is used as an electrode for measuring a bio-signal. Further, one or more embodiments of the present disclosure can provide biometric information on various items with a single touch from the user because a plurality of electrodes and sensors are constantly touching the user because one or more embodiments of the present disclosure are worn by the user. Further, one or more embodiments of the present disclosure do not require a separate charging terminal because the electrodes that are used for measuring bio-signals can also be used as charging terminals. Further, according to one or more embodiments of the present disclosure, the electronic device does not require a separate magnetic or metallic member for coupling to a charging device because the electrodes that are used for measuring bio-signals can also be used as the metallic members that generate an attractive magnetic force with the charging device

The term “module” as used in this disclosure may refer to a certain unit that includes one of hardware, software and firmware or any combination thereof. The term module may be interchangeably used with unit, logic, logical block, component, or circuit, for example. The module may be the minimum unit, or part thereof, which performs one or more particular functions. The module may be formed mechanically or electronically. For example, the module disclosed herein may include at least one of ASIC (application-specific integrated circuit) chip, FPGAs (field-programmable gate arrays), and programmable-logic device, which have been known or are to be developed.

It will be understood that the above-described embodiments are examples to help easy understanding of the contents of the present disclosure and do not limit the scope of the present disclosure. Accordingly, the scope of the present disclosure is defined by the appended claims, and it will be construed that all corrections and modifications derived from the meanings and scope of the following claims and the equivalent concept fall within the scope of the present disclosure.

The above-described embodiments of the present disclosure can be implemented in hardware, firmware or via the execution of software or computer code that can be stored in a recording medium such as a CD ROM, a Digital Versatile Disc (DVD), a magnetic tape, a RAM, a floppy disk, a hard disk, or a magneto-optical disk or computer code downloaded over a network originally stored on a remote recording medium or a non-transitory machine readable medium and to be stored on a local recording medium, so that the methods described herein can be rendered via such software that is stored on the recording medium using a general purpose computer, or a special processor or in programmable or dedicated hardware, such as an ASIC or FPGA. As would be understood in the art, the computer, the processor, microprocessor controller or the programmable hardware include memory components, e.g., RAM, ROM, Flash, etc. that may store or receive software or computer code that when accessed and executed by the computer, processor or hardware implement the processing methods described herein. No claim element herein is to be construed under the provisions of 35 U.S.C. 112, sixth paragraph, unless the element is expressly recited using the phrase “means for.” In addition, an artisan understands and appreciates that a “processor” or “microprocessor” may be hardware in the claimed disclosure. Under the broadest reasonable interpretation, the appended claims are statutory subject matter in compliance with 35 U.S.C. §101.

What is claimed is:

1. An electronic device comprising:
a housing having a top surface and a bottom surface;
a first electrode disposed on the top surface of the housing, the first electrode electrically isolated from rest of the top surface;
a second electrode disposed on the bottom surface of the housing and contacting a user's body when the electronic device is worn by the user;
a sensor module electrically coupled to the first electrode and the second electrode;
a processor electrically coupled to the sensor module;
a display module electrically coupled to the processor; and
a memory electrically coupled to the processor,
wherein the memory stores instructions that, when executed by the processor, causes the sensor module to measure the user's bio-signal using the first electrode and the second electrode, and the processor to provide the user's health state information by analyzing the measured bio-signal.

2. The electronic device of claim 1, wherein the first electrodes comprises two sub-electrodes, the two sub-electrodes have substantially same area, and are disposed within a distance so that the user can simultaneously touch the two sub-electrodes with a finger.

3. The electronic device of claim 2, wherein the processor provides ECG (Electrocardiogram) information when one of the two sub-electrodes is touched by the user, and provides BIA (Bio-Impedance Analysis) information when both of the two sub-electrodes are simultaneously touched by the user.

4. The electronic device of claim 1, further comprising:
a first heart rate sensor module disposed on the top surface of the housing and configured to measure a heart rate of the user, an intensity of infrared rays in an environment of the user, and a degree of oxygen saturation in the user's blood.

5. The electronic device of claim 4, wherein the processor provides blood pressure information by analyzing bio-signals measured by the first electrode, the second electrode, and the first heart rate sensor module.

6. The electronic device of claim 4, further comprising:
a second heart rate sensor module disposed on the bottom surface of the housing and configured to measure a blood flow, a blood pressure change, and an heart rate of the user when the electronic device is worn by the user;
an RGB sensor disposed on a side of the top surface of the housing and configured to measure a light intensity in the environment of the user; and
a temperature sensor disposed on the bottom surface of the housing and configured to measure a body temperature of the user.

7. The electronic device of claim 6, wherein the temperature sensor is located on a surface of the second electrode that is opposite of an exterior of the electronic device.

8. The electronic device of claim 6, wherein the processor provides stress and emotion analysis information by analyzing a galvanic skin response measured by the second electrode when the electronic device is worn by the user, and provides sleep analysis information by analyzing blood flow information measured by the second heart rate sensor module and blue light information measured by the RGB sensor.

9. The electronic device of claim 1, wherein the first electrode comprises:
an outer surface exposed to an exterior of the electronic device; and
a contact surface extended from a side of the outer surface and configured to be in electrical contact with a printed circuit board.

10. The electronic device of claim 9, further comprising:
a first connecting element configured to connect the contact surface to the printed circuit board.

11. The electronic device of claim 1, further comprising:
a second connecting element disposed between the second electrode and a printed circuit board, and configured to electrically connect the second electrode to the printed circuit board.

12. The electronic device of claim 1, wherein the second electrode is electrically connected to a charging terminal of an external device when the external device is coupled to the electronic device for charging the electronic device.

13. The electronic device of claim 12, wherein the second electrode is made of a metallic or magnetic material that is attracted to a magnetic member disposed in the external device.

14. The electronic device of claim 13, further comprising:
at least one sensor configured to identify the magnetic member.

15. The electronic device of claim 13, wherein the processor identifies a coupling direction of the external device by using two sensors configured to identify the magnetic member, and switches a charging path that includes the second electrode based on the identification.

16. The electronic device of claim 12, wherein the external device further comprises:
a housing;
two contact pins exposed to an exterior of the housing and configured to contact the second electrode;
at least one magnetic member disposed in the housing and provides an attracting force to the second electrode;
an interface unit connected to a charging device for charging the electronic device; and
a printed circuit board coupled to the interface unit and the contact pin.

17. The electronic device of claim 16, wherein at least one of the contact pins is a pogo pin.

18. The electronic device of claim 16, wherein the two contact pins are disposed symmetrically at a right side and a left side of the external device.

19. The electronic device of claim 16, wherein the external device includes two magnetic members disposed relatively adjacent to the two contact pins.

20. The electronic device of claim 19, wherein the two magnetic members have different magnetic properties when the two magnetic members are disposed symmetrically at a right side and a left side of the external device.
